(19) United States
(12) Patent Application Publication
(10) Pub. No.: US 2025/0044318 A1
(43) Pub. Date: Feb. 6, 2025

(54) SMART DYNAMIC GESTURE STABILIZATION FOR COSMETIC APPLICATOR CONFIGURED FOR USERS WITH LIMITED MOBILITY

(71) Applicant: L'OREAL, Paris (FR)

(72) Inventors: Casey BARBARINO, San Anselmo, CA (US); Gregoire CHARRAUD, Jersey City, NJ (US); Hyosik Dennis MIN, Palisades Park, NJ (US)

(73) Assignee: L'OREAL, Paris (FR)

(21) Appl. No.: 18/228,336

(22) Filed: Jul. 31, 2023

(51) Int. Cl. G01P 13/00; A45D 44/00

(52) U.S. Cl. CPC G01P 13/00; A45D 44/00; A45D 2200/10; A61B 5/1101

(57) ABSTRACT

A motion stabilization device is provided for stabilization of a cosmetic applicator. The motion stabilization device includes a motion stabilizer handle configured to receive an adapter that holds a cosmetic applicator for a cosmetic application; and processing circuitry configured to detect a transition from a set up phase to an application phase of the cosmetic application process, and hold the cosmetic application in a set position when the transition is detected.

### FIELD

The present disclosure describes a system and features related to a device for modifying, mitigating, altering, reducing, compensation for, or the like, the movement of a cosmetic applicator caused by unintentional movements, tremors, limited mobility, or the like of a user.

### BACKGROUND

Unintentional movements of the human body, or human tremors, can occur in individuals suffering from motion disorders or even healthy individuals. Due to these unintentional movements, a person may have difficulty in performing a task that requires care and precision, such as applying a cosmetic composition to a part of the body, such as the face, hands, or feet.

Therefore, there is a need for a solution that allows application of a cosmetic composition that is compatible with the diverse and disposable nature of cosmetic applicators.

### SUMMARY

In an embodiment, a motion stabilization device is provided for stabilization of a cosmetic applicator, comprising: a motion stabilizer handle configured to receive an adapter that holds a cosmetic applicator for a cosmetic application; and processing circuitry configured to detect a transition from a set up phase to an application phase of the cosmetic application process, and hold the cosmetic application in a set position when the transition is detected.

In an embodiment, the processing circuitry is configured to detect the transition based on a predetermined change in movement patterns of the cosmetic applicator.

In an embodiment, the motion stabilization device further includes a force sensor, wherein the processing circuitry is configured to detect the transition based on a detected threshold amount of force placed upon the cosmetic applicator.

In an embodiment, the motion stabilization device further includes a torque sensor, wherein the processing circuitry is configured to detect the transition based on a detected threshold amount of torque placed upon the cosmetic applicator.

In an embodiment, the adapter further includes a hall sensor, and the processing circuitry is configured to detect the transition based on the hall sensor detecting that a cosmetic applicator has been inserted into the adapter.

In an embodiment, the motion stabilization device further includes an embedded camera and/or proximity sensor, and the processing circuitry is configured to detect the transition based on the camera and/or proximity sensor detecting that the cosmetic applicator has moved towards a target area of the cosmetic application.

In an embodiment, the motion stabilization device further includes a microphone, and the processing circuitry is configured to detect the transition based on a audible input from the user indicating that the application phase has started.

In an embodiment, the motion stabilization device further includes a switch, and the processing circuitry is configured to detect the transition based on an activation of the switch by the user indicating that the application phase has started.

In an embodiment, the processing circuitry is configured to detect the transition based on detecting that the motion stabilization device is held in a singular position for a threshold amount of time.

In an embodiment, the processing circuitry is configured to provide a learning mode to learn the predetermined change in movement patterns specific to the user, wherein in the learning mode the processing circuitry is configured to: (i) record movement patterns before the application phase has started (ii) receive an input from the user directly indicating when the application phase has started and record movement patterns during the application phase; (iii) receive an input from the user directly indicating that the application phase has ended and record movement patterns for after the application phase has ended.

### BRIEF DESCRIPTION OF THE DRAWINGS

A more complete appreciation of the embodiments and many of the attendant advantages thereof will be readily obtained as the same becomes better understood by reference to the following detailed description when considered in connection with the accompanying drawings, wherein:

FIG. 1 shows a motion stabilizing device.

FIG. 2 shows how the motion stabilizing device couples with an adaptor and a make-up applicator.

FIG. 3A shows a diagram of the internal components of motion stabilizing device.

FIG. 3B shows a diagram of an alternative embodiment of the motion stabilizing device in which the receiver portion includes an electromagnetic positioner instead of the motive elements shown in FIG. 3A.

FIG. 4 shows an overview of a universal adapter handle connection system.

FIG. 5A depicts a situation in which the motion stabilizer is in a setup phase of using the motion stabilizer device.

FIG. 5B depicts a situation in the motion stabilizer is an application phase

FIG. 5C shows different phases of the process which are applicable to the above-described device.

FIG. 6 shows a flowchart of a process in which the motion stabilizer detects when the user has transitioned from the setup phase to the application phase.

FIG. 7A shows one option in which an actual movement pattern of handling the motion stabilizer is analyzed and when the user's movements match expected movements for the application phase.

FIG. 71B shows a scenario in which at a time region indicates a particular change in the measurements of the X, Y, and Z sensors in comparison to the application phase.

FIG. 8A depicts a situation in which force is applied by the user to the flexible portion of the motion stabilizer handle to cause the cosmetic applicator to orient in the direction desired for cosmetic application.

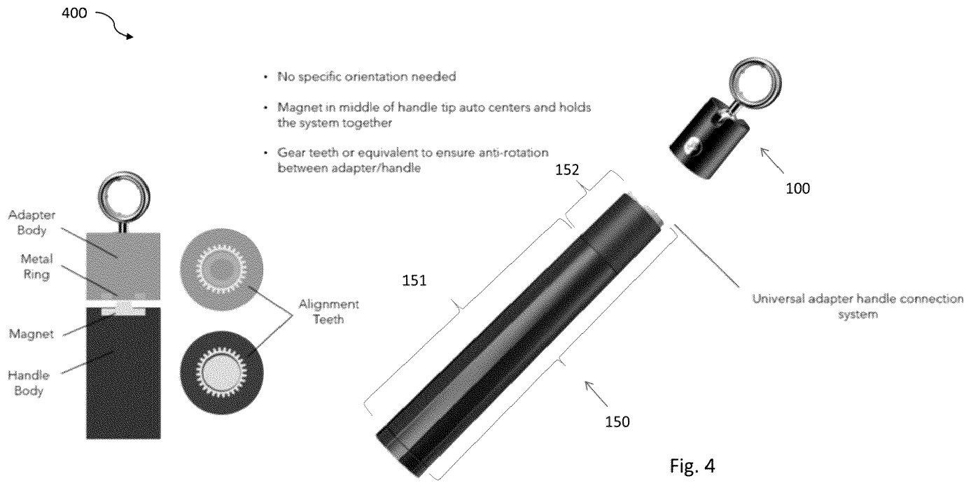

FIG. 8B shows a situation when the cosmetic adapter depicted in FIG. 4 is being used.

FIG. 9A shows different components of the motion stabilizer and adapter.

FIG. 9B shows components included within the receiver portion of the motion stabilizer.

FIG. 9C-9D show how the proximity sensor is used to determine a phase.

FIG. 10 shows a flowchart according to a method of learning user's movement patterns.

FIG. 11 is a diagram shown how machine learning can help determine movement patterns which indicate a particular phase of use.

FIG. 12 shows the usage of the deep learning model after training has reached an adequate level.

FIG. 13 shows a system that includes a mobile user device, a motion stabilizer, and a server device.

FIG. 14 shows hardware components of a mobile user device.

FIG. 15 shows hardware components of a server device.

### DETAILED DESCRIPTION

The present disclosure describes a cosmetic applicator system that minimizes modifies, mitigates, alters, reduces, compensates for, or the like unintentional movements by stabilizing, orienting, operating, controlling, etc. an applicator for a user and is also designed to be flexible to accommodate different types of commercially available cosmetic applications. The present disclosure further describes a system and features to enhance the functionality of such a cosmetic applicator system.

The basic features and operation of a motion stabilizing device for a cosmetic applicator is described in U.S. Pat. No. 11,458,062, which is incorporated herein by reference.

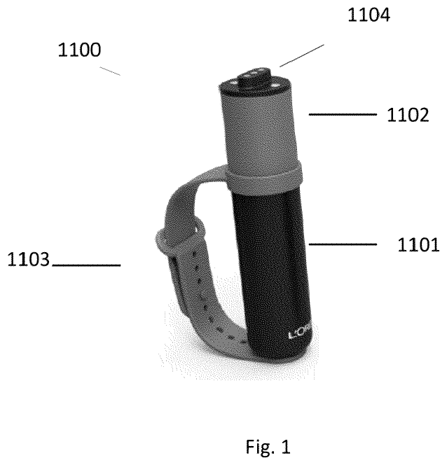

FIG. 1 shows a conventional motion stabilizing device 1100, which serves as a base unit for receiving a cosmetic applicator according to an embodiment. The device 1100 includes a handle portion 1101, a receiver portion 1102 and a strap 1103. The receiver portion 1102 includes an interface 1104, shown as a male connector that couples with a cosmetic applicator, which will be discussed in detail below. The receiver portion could be utilized for communication between the base unit and the applicator. The connection to an adaptor and/or an applicator could be accomplished with a mechanical coupling, such as screw-in or snap-fit, or it could be accomplished with magnets.

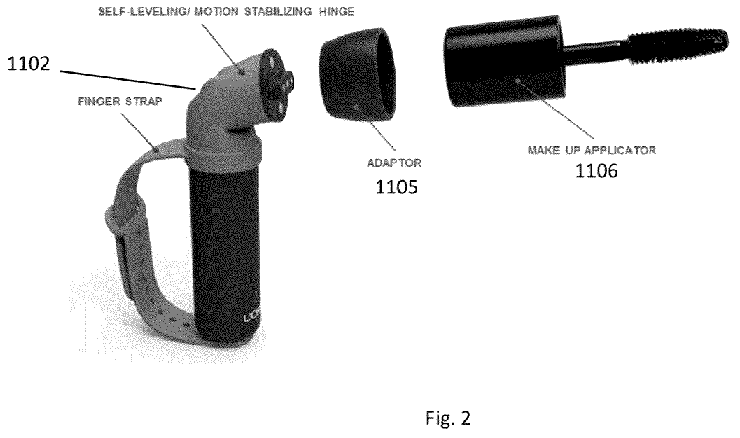

FIG. 2 shows how the device 1100 couples with an adaptor 1105 and a make-up applicator 1106. It can be seen that the adaptor fits over the exposed end of the receiver portion 1102. The adaptor includes electrical mating connectors (a female connector—not shown) in a recessed portion to make contact with the electric interface of the receiver portion 1101.

As shown in FIG. 2, the receiver portion 1102 is configured to contort, articulate, reposition, etc., between an upright posture (as shown in FIG. 1) and an angled posture (as shown in FIG. 2). This is accomplished with a hinge mechanism contained inside the receiver portion 1102. FIG. 2 shows that the hinge mechanism is a self-leveling/motion stabilizing hinge.

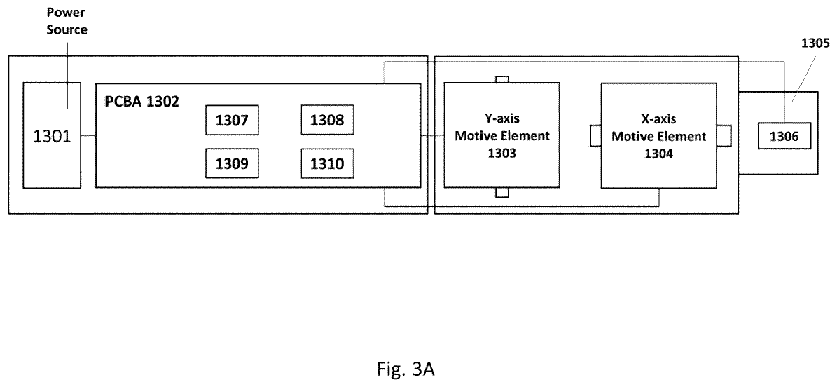

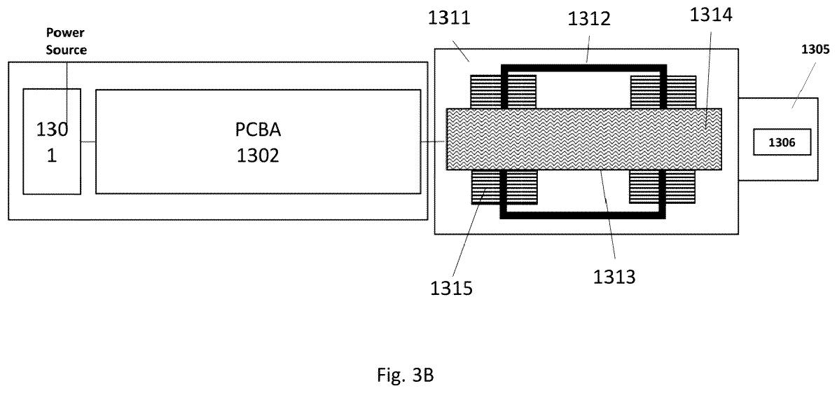

FIG. 3A shows a diagram of the internal components of device 1100 according to one embodiment. In the handle portion, the device includes a power source 1301, which may be a battery or the like. The device includes a printed circuit board assembly (PCBA) 1302, which may include positional sensor circuitry 1307, reader circuitry 1308, control circuitry 1309, and communication interface 1310, as understood in the art.

For instance, as the sensor circuitry 1307, the PCBA may include at least one inertial sensor and at least one distributed motion sensor to detect unintentional muscle movements and measure signals related to these unintentional muscle movements that are created when a user adversely affects motion of the applicator. These sensors also detect the motion of the stabilized output relative to device. The control circuitry sends voltage commands in response to the signals to the motion generating elements (described below) to cancel the user's tremors or unintentional muscle movements. This cancellation maintains and stabilizes a position of the applicator, keeping it stable.

One of ordinary skill in the art readily recognizes that a system and method in accordance with the present invention may utilize various implementations of the control circuitry and the sensor circuitry and that would be within the spirit and scope of the present invention. In one embodiment, the control circuitry 1309 comprises an electrical system capable of producing an electrical response from sensor inputs such as a programmable microcontroller or a field-programmable gate array (FPGA). In one embodiment, the control circuitry comprises an 8-bit ATMEGA8A programmable microcontroller manufactured by Atmel due to its overall low-cost, low-power consumption and ability to be utilized in high-volume applications.

In one embodiment, the at least one inertial sensor in the sensor circuitry is a sensor including but not limited to an accelerometer, gyroscope, or combination of the two. In one embodiment, the at least one distributed motion sensor in the sensor circuitry is a contactless position sensor including but not limited to a hall-effect magnetic sensor.

The system created by the combination of the sensor circuitry, the control circuitry, and the motion generating elements may be a closed-loop control system that senses motion and acceleration at various points in the system and feeds detailed information into a control algorithm that moves the motion-generating elements appropriately to cancel the net effect of a user's unintentional muscle movements and thus stabilize the position of the applicator. The operation and details of the elements of the control system and control algorithm are understood in the art, as described in U.S. PG Publication 2014/0052275A1, incorporated herein by reference.

The communication interface 1310 may include a network controller such as BCM43342 Wi-Fi, Frequency Modulation, and Bluetooth combo chip from Broadcom, for interfacing with a network.

In the receiver portion of the device, there may be two motive elements to allow 3-dimensional movement of the receiver as anti-shaking movement. The two motive elements include a y-axis motive element 1303 and an x-axis motive element 1304, each being connected to and controlled by the PCBA 1302. Each of the motive elements may be servo motors as understood in the art. The device further includes end effector coupling 1305, which is configured to couple with the adaptor 1105. The end effector coupling 1305 may include a radiofrequency identification (RFID) reader 1306, configured to read an RFID tag, which may be included with the applicator, as will be discussed below.

FIG. 3B shows a diagram of an alternative embodiment of the device 1100 in which the receiver portion includes an electromagnetic positioner 1311 instead of the motive elements shown in FIG. 3A. The electromagnetic positioner 1311 may include U-shaped magnetic cores 1312 arrayed around a non-magnetic tube 1313, which is filled with a magnetic fluid 1314. Each of the magnetic cores has arm portions that are surrounded by windings 1315. The magnetic cores may be controlled by the control circuitry in the PCBA 1302 to act as a controllable active magnetic field-generating structure which is used to generate a variable magnetic field that acts upon the magnetic fluid, causing it to be displaced, thereby enabling the armature to be moved to a desired coordinate position and/or orientation. The details of implementing the electromagnetic positioner 1311 may be found in U.S. Pat. No. 6,553,161, which is incorporated herein by reference.

In the above-described conventional motion stabilizing device, there is a problem that the interface 1104 that receives the adaptor 1105 requires a specific point of attachment to align properly with the interface.

Therefore, the below embodiments provide a universal adapter connection between the handle of the motion stabilizing device in order to improve user experience and reduce the struggle and time taken to set up the system for use.

In one embodiment, the present disclosure is directed towards a cosmetic applicator. The cosmetic applicator can be used for a variety of cosmetics and cosmetic applications, including, but not limited to, mascara, eyeliner, eyebrow products, lip products (lipstick, lip gloss, lip liner, etc.), skin products, and/or hair products. In one embodiment, the cosmetic applicator can include an adapter, wherein the adapter can connect the cosmetic applicator to a motion stabilizer. The motion stabilizer can be, for example, a handle that can counteract unintentional motions such as tremors or spasms. These motions can interfere with the application of cosmetics and can also make it difficult to generally interact with cosmetic applicators or tools. For example, the many cosmetic products require a twisting motion or force to be applied to open or extrude the product. It can be difficult for users to achieve the range of motion or the precision necessary to apply these forces to the cosmetic. In one embodiment, the cosmetic applicator can hold a cosmetic and can enable the proper force to be applied to the cosmetic to open, close, mix, stir, blend, extrude, or achieve other similar functions necessary for application.

FIG. 4 shows an overview of a universal adapter handle connection system 400. The basic features and operation of universal adapter handle connection system 400 are described in co-pending U.S. application Ser. Nos. 18/091,882; 18/091,920; 18/091,843; 18/091,925; 18/148,957; 18/148,880; and 18/148,930, which are incorporated herein by reference. The system includes a motion stabilizer device 150 that includes a handle portion 151 and a hinge portion 152 (receiver portion) that is functionally similar to the device 1100 shown in FIG. 1. It further includes a universal adapter 100 that attaches to the device 150 and also holds different types of cosmetic applicators.

Currently, the above-described device does not know when the user is applying makeup or setting up positioning. This can cause difficulty of controlling the device position once they start applying makeup since the device will sometimes continue to move when they don't intend for it to move (i.e. want it to stay still in space once they start applying). The device should understand when the user is transitioning from setup of device orientation to actually applying the makeup.

This below features provide a solution to detect (either automated or by user input) when the user is transitioning from setup to application. When application is detected, the device will pause/hold its orientation in space to allow for accurate and controlled application by the user. This doesn't mean that the device will be held static in a fixed state, but rather that the position of the applicator will be attempting to hold still in space while the motion stabilizer makes constant adjustments to account for the unintentional movements of the user's hands.

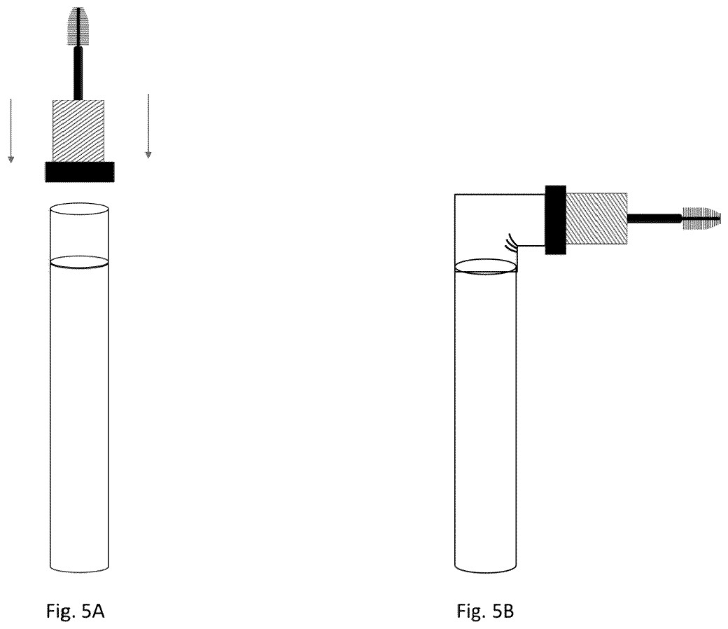

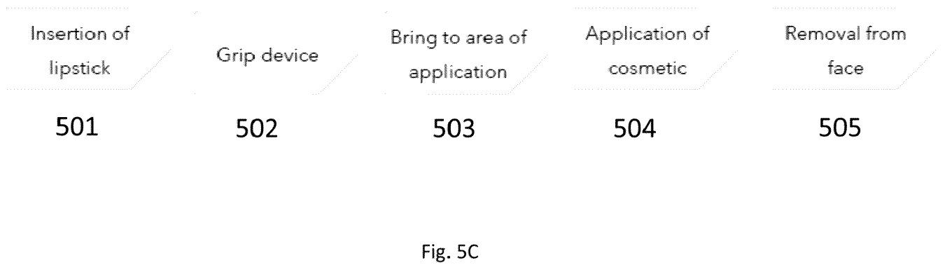

FIG. 5A depicts a situation in which the motion stabilizer is in a setup phase of using the motion stabilizer device. In this phase, the user is possibly attempting to attach the cosmetic applicator to the motion stabilizer and does not yet want the motion stabilizer to be oriented and held in the position that is required for application of the cosmetic product.

FIG. 5B depicts a situation in the motion stabilizer is an application phase and the user is ready for the motion stabilizer to be oriented and held in the position required for application of the cosmetic product.

FIG. 5C shows different phases of the process which are applicable to the above-described device. Phase 501 involves the insertion of a cosmetic applicator (such as lipstick) into an adapter on the device. Phase 502 involves the user gripping the device to start to use the device. Phase 503 involves bringing the device to the target area of application (such as the lips of a user). Phase 504 involves the actual application of the cosmetic to the target area of the application. Finally phase 505 involves the removal of the applicator from the target area and the completion of the process.

The below embodiments provide a solution to detect (either automated or by user input) when the user is transitioning from setup to application. When application is detected, the device will pause/hold its orientation to allow for accurate and controlled application by the user. The device should understand when the user is transitioning from setup of device orientation to actually applying the makeup. Once this is detected, the device will hold its current orientation until application is complete or directed otherwise from the user.

The features described below achieve: (i) stable device positioning once application is started; (ii) improved confidence from user that device will do what they intend it to do; and (iii) potentially less user interaction, which improves accessibility for users that have trouble with fine motor skills or pain

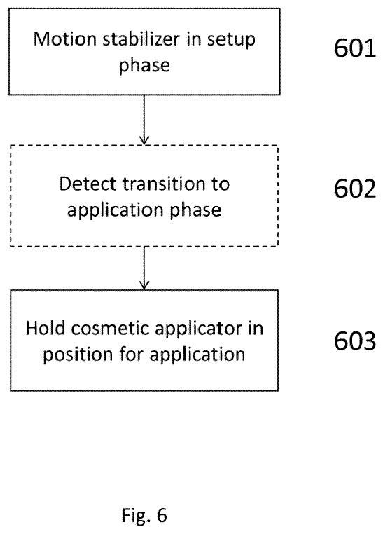

FIG. 6 shows a flowchart of a process in which the motion stabilizer detects when the user has transitioned from the setup phase to the application phase. Upon such detection, the motion stabilizer will hold the orientation of the cosmetic applicator in the proper position for cosmetic application (such as the position shown on FIG. 5B). In step 601, the motion stabilizer is in the setup phase, which may include the user lifting the motion stabilizer from a base unit and/or inserting the cosmetic applicator into the motion stabilizer. In step 602, the motion stabilizer detects that there is a transition from the setup phase to the application phase. Upon such detection, in step 603, the motion stabilizer will hold he general orientation of the cosmetic applicator in the user's desired position for make-up application. Different options for performing the detection step at step 602 will be described below.

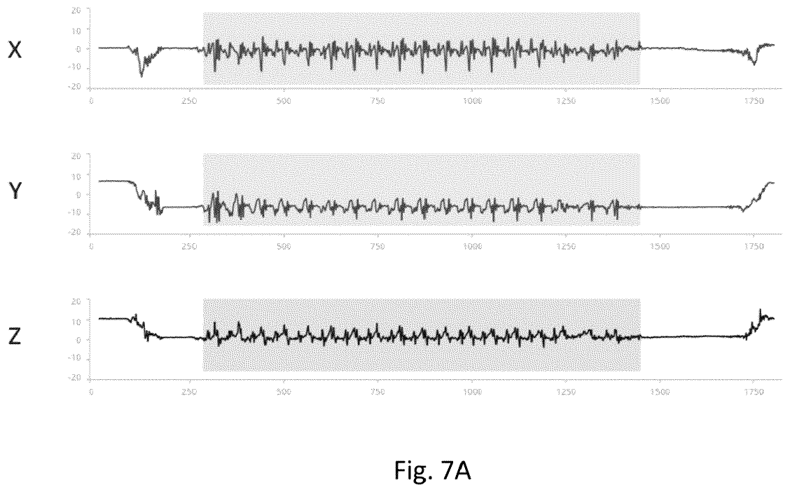

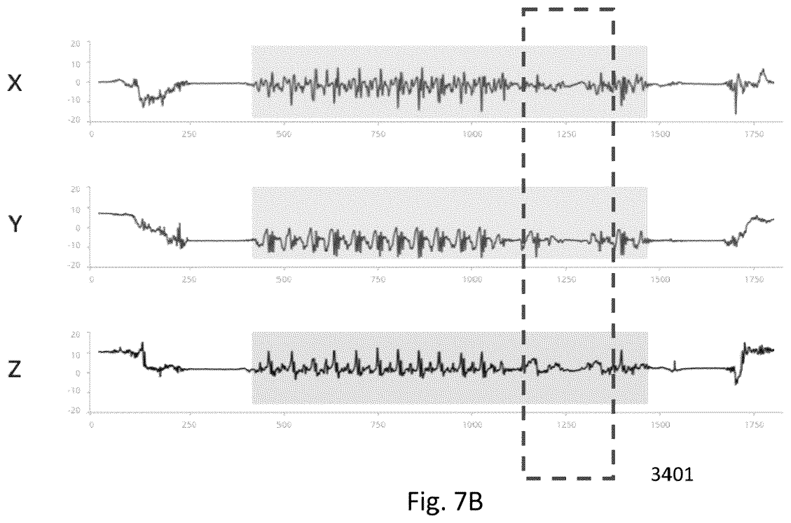

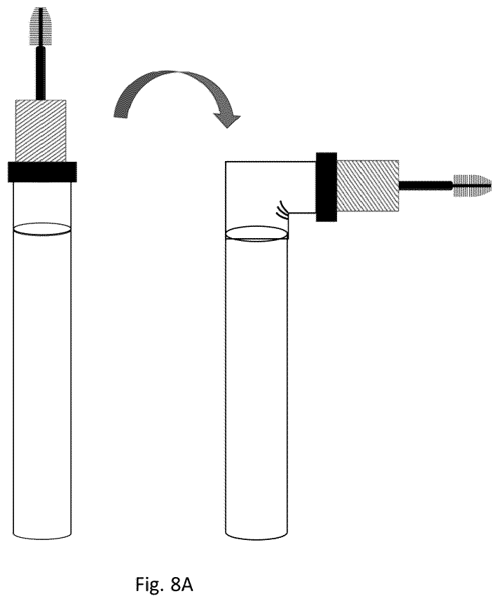

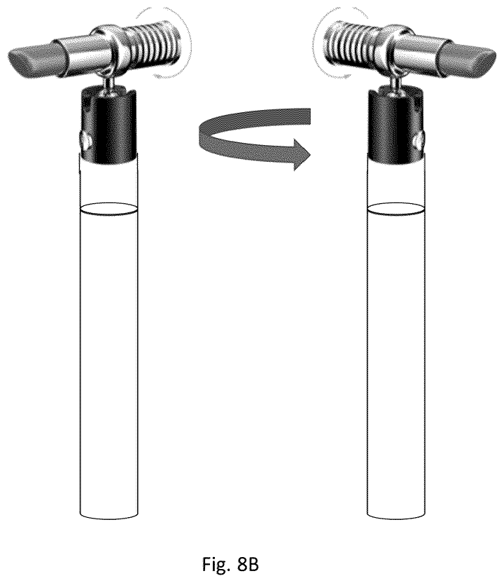

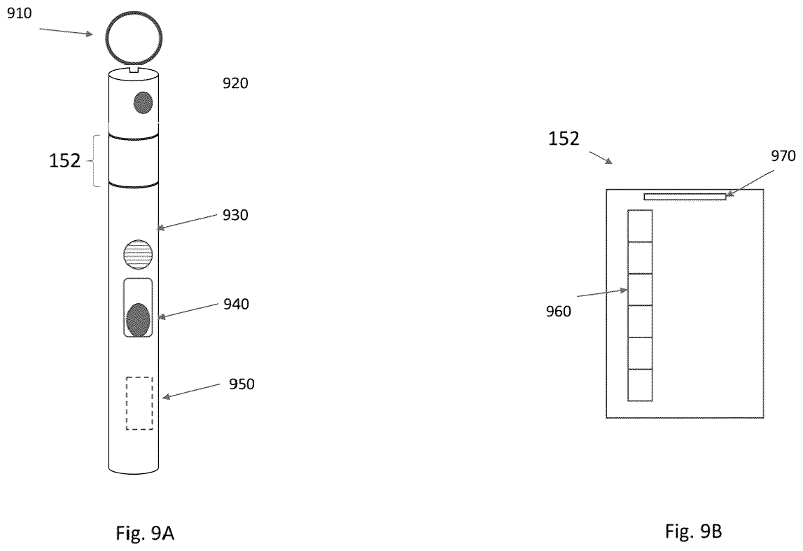

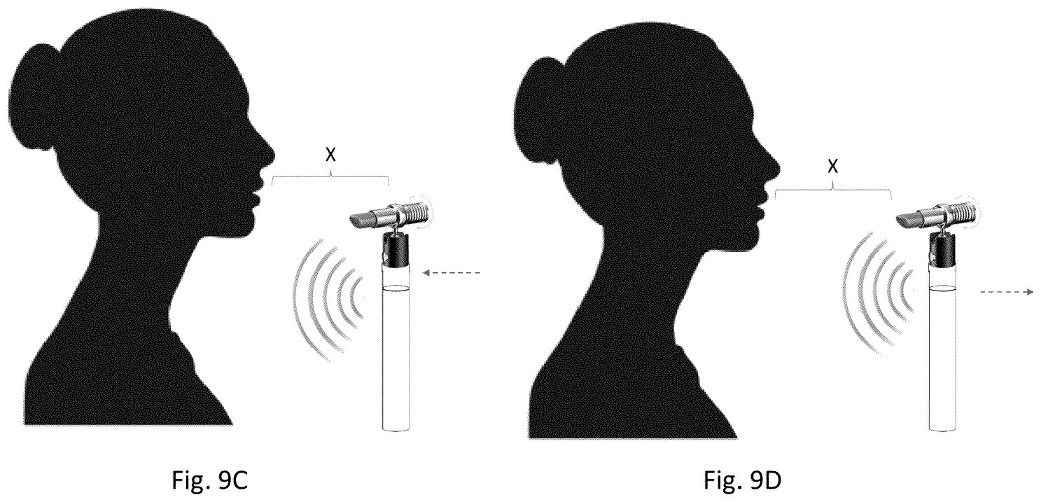

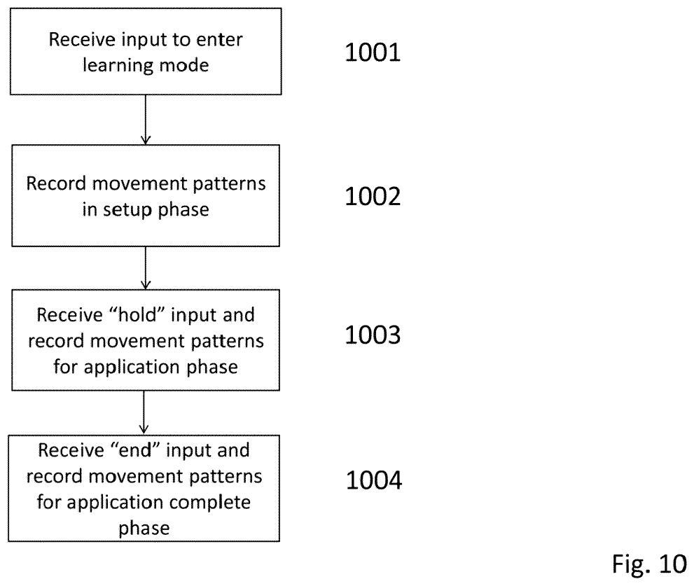

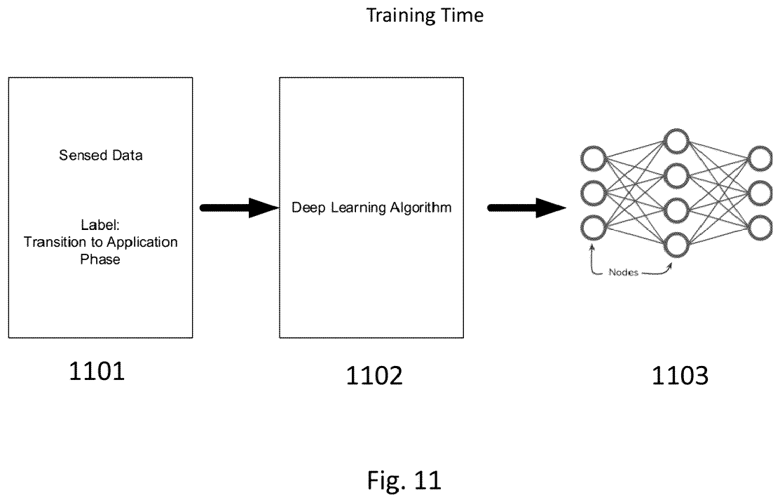

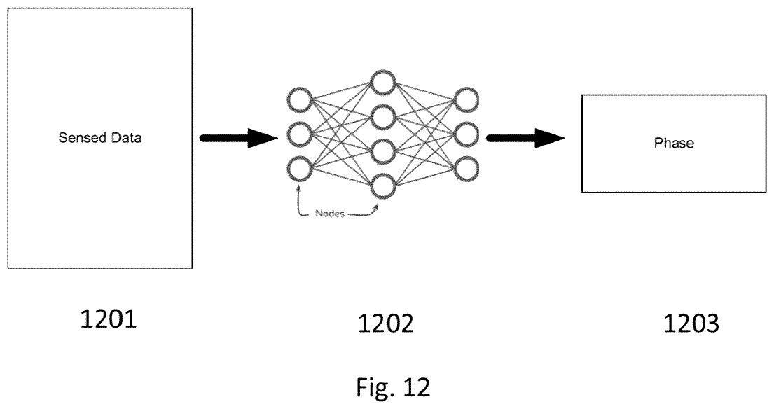

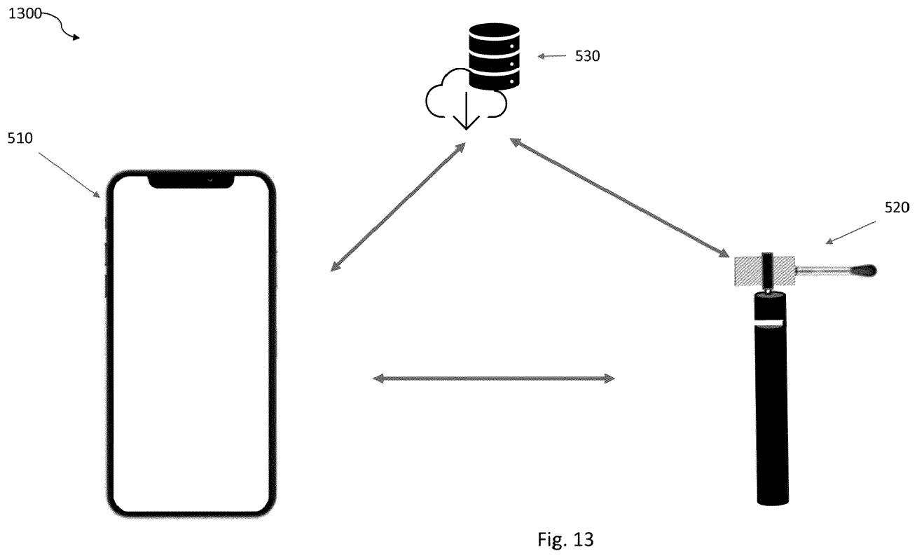

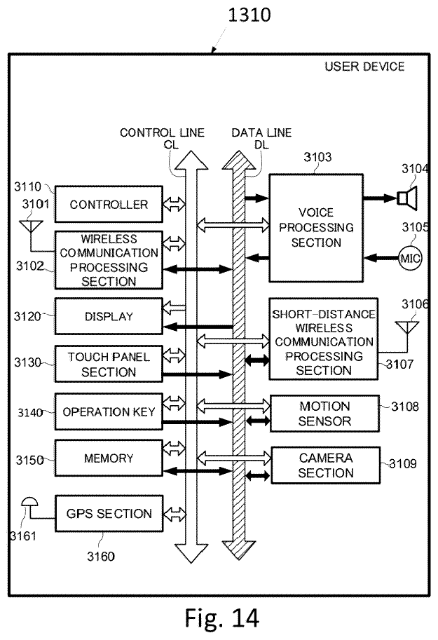

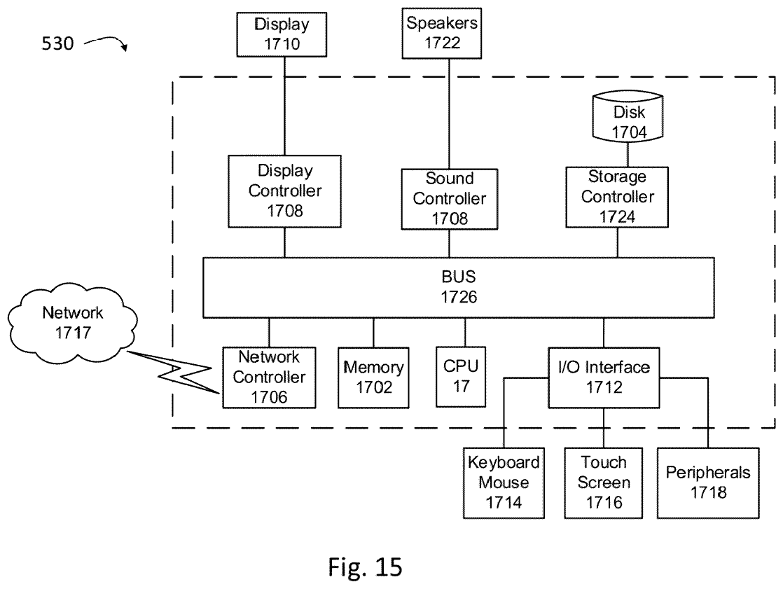

FIG. 7A shows one option in which an actual movement pattern of handling the motion stabilizer is analyzed and when the user's movements match expected movements for the application phase, then the transition to the application phase is detected. FIG. 7A shows example measurements taken by the accelerometer in the X, Y. and Z directions during movement of the motion stabilizer during a setup phase.

FIG. 7B shows a scenario in which at a time region indicated by 3401 indicates a particular change in the measurements of the X, Y, and Z sensors in comparison to the application phase. Based on type of change, the motion stabilizer detects that the application phase has started.

FIG. 8A depicts a situation in which force is applied by the user to the flexible portion of the motion stabilizer handle to cause the cosmetic applicator to orient in the direction desired for cosmetic application. A force or torque sensor may be included in the motion stabilizer which detects a threshold amount of force or torque which will indicate that the transition to the application phase is detected.

FIG. 88 shows a situation when the cosmetic adapter 100 depicted in FIG. 4 is being used. In this situation, there might not be any force which bends the flexible portion of the motion stabilizer a significant amount, but instead the applicator may be twisted to be in the desired position. In this situation, a torque sensor is used to determine if a threshold amount of torques has been applied, which will indicate that the transition to the application phase is detected.

FIG. 9A shows a situation in which the cosmetic adapter 100 includes a hall effect sensor 910 which can detect the presence of a cosmetic applicator that has been inserted into the adapter. With this type of sensor, the application phase may be detected based solely on insertion of the applicator if normally no other adjustments are made after insertion.

FIG. 9A further shows that the cosmetic adapter 100 includes an embedded camera and/or proximity sensor 920. With this type of sensor, detection of the application phase may be detected based on a combination of insertion of the applicator into the adapter and movement of the adapter towards the user.

FIG. 9A further shows that the motion stabilizer may include means to allow the user to directly input that the application phase has started. For instance, the motion stabilizer includes a microphone 930, that will allow the user to enter voice commands to “hold” the applicator at the current position for the application phase. Alternatively, the motion stabilizer includes a slider switch 940, that will allow the user to directly toggle the motion stabilizer to hold its position for the application phase.

FIG. 9A further shows that the motion stabilizer includes timer circuitry 950. The timer can be used to enter a “hold” mode if the device is held in a singular position for a threshold amount of time (i.e., 3 seconds).

FIG. 9B shows additional details regarding the receiver portion 152. The receiver portion 152 includes flex sensor 960, as understood in the art, to detect forced flexing of the receiver portion when the user in the setup phase. The receiver portion further includes a torque/force sensor 970, as understood in the art, that can detect a force to twist or turn the adapter that is disposed on the receiver portion 152.

FIGS. 9C and 9D further illustrate how the embedded camera and/or proximity sensor 920 is used to detect the start of the application and the end of the application phase. FIG. 9C shows (for the lipstick example) that the sensor 920 detects when the device is brought within X inches of a user, which can be used to detect the start of the application phase. FIG. 9D shows that the sensor further detects (after the application phase started) that the device is moved outside of the range of X inches from the user, which can be used to detect the end of the application phase.

Additionally, it was noted above for FIGS. 7A and 7B, that a change in the movement patterns may be detected to determine when an application phase is started. Related to this feature, the device can learn from a diagnosis process to determine a user's profile (i.e. types of movements made for their disability) and automatically recognize by means of their movements that application is being attempted/started.

For instance, FIG. 10 shows a flowchart according to this method of learning. In Step 1001, the motion stabilizer may be set to “learning mode”, in which the user utilizes voice commands or the slider switch 940 to indicate when the application process has started. During a predetermined number of uses, the motion stabilizer will record the movement patterns before the application phase has started in the setup phase (step 1002) and during the application phase after the user inputs the switch or voice command to hold the applicator in the proper configuration (step 1003). The user can also enter a command of “hold off” (or the like) or disable the slider switch to indicate that the application has ended, and the device will record the movement patterns indicating the application phase has ended (step 1004).

While the above example describes a situation in which the user enters a learning phase to have their own movement patterns learned, the user may also avoid the learning phase and utilize a “crowd-sourcing” technique that leverages the movement patterns of other users that have a similar profile or characteristics.

FIG. 11 is a diagram shown how machine learning can help determine movement patterns which indicate a particular phase of use. During training, previous users data is collected that has sensed data as described above with a label corresponding to the phase of use. For instance, a sensed movement pattern representing the transition to the application phase may be inputted. However, any of the data sensed from the above-noted sensing mechanisms may b used. These inputs are provided at stage 1101, with the label of the phase (application phase in this example). While the application phase is used in this example, data corresponding to any of the phases (such as setup or post-application) may be uses and labeled appropriately.

The inputs are provided to a deep learning algorithm in step 1102. The deep learning algorithm used may be based on available software as known in the art, such as Tensorflow, Keras, Mxnet, Caffe, or Pytorch. The result of the labeled training will be a neural network at step 1103. The neural network created includes nodes of each layer are clustered, the clusters overlap, and each cluster feeds data to multiple nodes of the next layer.

FIG. 12 shows the usage of the deep learning model after training has reached an adequate level. This is referred to as “inference time” since recommendation will be inferred from input data without a label. It can be seen that the input stage does not include a label of phase itself. The inputs of data sensed from a user are fed to the trained neural network at step 1202, which will provide an output at step 1203 of the phase that best fits the inputted data. With the neural network model described herein, complex diagnosis results can be utilized to determine a custom motion stabilization profile for a user without having to come with up predetermined manual settings.

FIG. 13 shows a system 1300 according to an embodiment includes a mobile user deice 1310 and the motion stabilizer 1320 described above. The system further includes a cloud server device 1330 that connects to both the smartphone and the motion stabilizer. The smartphone may be communicatively coupled with the motion stabilizer and receive any of the data captured by the motion stabilizer as was described above. The smartphone may transmit data to the server device as necessary. For instance, server device may be utilized to analyze the data and make determinations based on the data, such as any of the determinations on the current phase, as was describe above.

Furthermore, while the above-described embodiments describe the functionality being performed by a server device 1330, it will be appreciated that the functionality can be performed by the user device 1310 or a personal computer (not shown) of the user.

FIG. 14 is a more detailed block diagram illustrating a mobile user device 1310 according to certain embodiments of the present disclosure. In certain embodiments, user device 1310 is a smartphone. However, the skilled artisan will appreciate that the features described herein may be adapted to be implemented on other devices (e.g., a laptop, a tablet, a server, an e-reader, a camera, a navigation device, etc.). The exemplary user device 1310 includes a controller 3110 and a wireless communication processor 3102 connected to an antenna 3101. A speaker 3104 and a microphone 3105 are connected to a voice processor 3103.

The controller 3110 may include one or more Central Processing Units (CPUs), and may control each element in the user device 1310 to perform functions related to communication control, audio signal processing, control for the audio signal processing, still and moving image processing and control, and other kinds of signal processing. The controller 3110 may perform these functions by executing instructions stored in a memory 3150. Alternatively or in addition to the local storage of the memory 150, the functions may be executed using instructions stored on an external device accessed on a network or on a non-transitory computer readable medium. As described above in relation to FIG. 16, the controller 3110 may execute instructions allowing the controller 3110 to function as the display control unit 3211, operation management unit 3212 and game management unit 3213 depicted in FIG. 14.

Next, a hardware description of the server device 1330 according to exemplary embodiments is described with reference to FIG. 15. In FIG. 15, the server device 1330 includes a CPU 1700 which performs the processes described above/below. The process data and instructions may be stored in memory 1702. These processes and instructions may also be stored on a storage medium disk 1704 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the [device]communicates, such as a server or computer.

Further, the claimed advancements may be provided as a utility application, background daemon, or component of an operating system, or combination thereof, executing in conjunction with CPU 1700 and an operating system such as Microsoft Windows 7, UNIX, Solaris, LINUX, Apple MAC-OS and other systems known to those skilled in the art.

The hardware elements in order to achieve the [device] may be realized by various circuitry elements, known to those skilled in the art. For example, CPU 1700 may be a Xenon or Core processor from Intel of America or an Opteron processor from AMD of America, or may be other processor types that would be recognized by one of ordinary skill in the art. Alternatively, the CPU 1700 may be implemented on an FPGA, ASIC, PLA) or using discrete logic circuits, as one of ordinary skill in the art would recognize. Further, CPU 1700 may be implemented as multiple processors cooperatively working in parallel to perform the instructions of the inventive processes described above.

The [device] in FIG. 15 also includes a network controller 1706, such as an Intel Ethernet PRO network interface card from Intel Corporation of America, for interfacing with network 1717. As can be appreciated, the network 1717 can be a public network, such as the Internet, or a private network such as an LAN or WAN network, or any combination thereof and can also include PSTN or ISDN sub-networks. The network 1717 can also be wired, such as an Ethernet network, or can be wireless such as a cellular network including EDGE, 30 and 4G wireless cellular systems. The wireless network can also be WiFi, Bluetooth, or any other wireless form of communication that is known.

The [device] further includes a display controller 1708, such as a NVIDIA GeForce GTX or Quadro graphics adaptor from NVIDIA Corporation of America for interfacing with display 1710, such as a Hewlett Packard HPL2445w LCD monitor. A general purpose I/O interface 1712 interfaces with a keyboard and/or mouse 1714 as well as a touch screen panel 1716 on or separate from display 1710. General purpose I/O interface also connects to a variety of peripherals 1718 including printers and scanners, such as an OfficeJet or DeskJet from Hewlett Packard.

A sound controller 1720 is also provided in the [device], such as Sound Blaster X-Fi Titanium from Creative, to interface with speakers/microphone 1722 thereby providing sounds and/or music.

The general purpose storage controller 1724 connects the storage medium disk 1704 with communication bus 1726, which may be an ISA, EISA, VESA, PCI, or similar, for interconnecting all of the components of the [device]. A description of the general features and functionality of the display 1710, keyboard and/or mouse 1714, as well as the display controller 1708, storage controller 1724, network controller 1706, sound controller 1720, and general purpose I/O interface 1712 is omitted herein for brevity as these features are known.

The above-noted features will provide improved confidence from user that device will do what they intend it to do. By requiring potentially less user interaction, the above features improve accessibility for users that have trouble with fine motor skills or pain.

Obviously, numerous modifications and variations of the present invention are possible in light of the above teachings. It is therefore to be understood that within the scope of the appended claims, the invention may be practiced otherwise than as specifically described herein.

1. A motion stabilization device for stabilization of a cosmetic applicator, comprising:
a motion stabilizer handle configured to receive an adapter that holds a cosmetic applicator for a cosmetic application; and
processing circuitry configured to
detect a transition from a set up phase to an application phase of the cosmetic application process, and
hold the cosmetic application in a set position when the transition is detected.

2. The motion stabilization device according to claim 1, wherein the processing circuitry is configured to detect the transition based on a predetermined change in movement patterns of the cosmetic applicator.

3. The motion stabilization device according to claim 1, further comprising a force sensor, wherein the processing circuitry is configured to detect the transition based on a detected threshold amount of force placed upon the cosmetic applicator.

4. The motion stabilization device according to claim 1, further comprising a torque sensor, wherein the processing circuitry is configured to detect the transition based on a detected threshold amount of torque placed upon the cosmetic applicator.

5. The motion stabilization device according to claim 1, wherein the adapter further includes a hall sensor, and the processing circuitry is configured to detect the transition based on the hall sensor detecting that a cosmetic applicator has been inserted into the adapter.

6. The motion stabilization device according to claim 1, further comprising an embedded camera and/or proximity sensor, and the processing circuitry is configured to detect the transition based on the camera and/or proximity sensor detecting that the cosmetic applicator has moved towards a target area of the cosmetic application.

7. The motion stabilization device according to claim 1, further comprising a microphone, and the processing circuitry is configured to detect the transition based on a audible input from the user indicating that the application phase has started.

8. The motion stabilization device according to claim 1, further comprising a switch, and the processing circuitry is configured to detect the transition based on an activation of the switch by the user indicating that the application phase has started.

9. The motion stabilization device according to claim 1, wherein the processing circuitry is configured to detect the transition based on detecting that the motion stabilization device is held in a singular position for a threshold amount of time.

10. The motion stabilization device according to claim 1, wherein the processing circuitry is configured to provide a learning mode to learn the predetermined change in movement patterns specific to the user, wherein in the learning mode the processing circuitry is configured to:
(i) record movement patterns before the application phase has started
(ii) receive an input from the user directly indicating when the application phase has started and record movement patterns during the application phase;
(iii) receive an input from the user directly indicating that the application phase has ended and record movement patterns for after the application phase has ended.

11. A method, implemented by processing circuitry of a motion stabilization device for stabilization of a cosmetic applicator, the motion stabilization device including a motion stabilizer handle configured to receive an adapter that holds a cosmetic applicator for a cosmetic application, the method comprising:
detecting a transition from a set up phase to an application phase of the cosmetic application process, and
holding the cosmetic application in a set position when the transition is detected.
